(19) United States
(12) Patent Application Publication
(10) Pub. No.: US 2004/0137067 A1
(43) Pub. Date: Jul. 15, 2004

(54) Adhesive applicator tip with a polymerization initiator, polymerization rate modifier, and/or bioactive material

(75) Inventors: Upvan Narang, Raleigh, NC (US); William M. Cotter, Raleigh, NC (US); Keith R. D'Alessio, Cary, NC (US); Anthony Sherbondy, Raleigh, NC (US); Gabriel N. Szabo, Raleigh, NC (US)

Correspondence Address: OLIFF & BERRIDGE, PLC, P.O. BOX 19928, ALEXANDRIA, VA, 22320, US

(73) Assignee: Closure Medical Corporation, Raleigh, NC

(21) Appl. No.: 10/705,916

(22) Filed: Nov. 13, 2003

Related U.S. Application Data
(63) Continuation-in-part of application No. 09/430,177, filed on Oct. 29, 1999.
(63) Continuation-in-part of application No. 09/069,979, filed on Apr. 30, 1998, now abandoned.

(51) Int. Cl. A61K 9/14

(52) U.S. Cl. 424/486

(57) ABSTRACT

An applicator tip for an applicator for applying a polymerizable monomeric adhesive composition can include a bioactive material, a flavorant, a polymerization initiator, and/or a polymerization rate modifier.

This application is a Continuation-In-Part of U.S. patent application Ser. No. 09/430,177, filed Oct. 29, 1999, the entire disclosure of which is incorporated herein by reference, which in turn is a Continuation-In-Part of U.S. patent application Ser. No. 09/069,979, filed Apr. 30, 1998, the entire disclosure of which is incorporated herein by reference.

### BACKGROUND OF THE INVENTION

1. Field of the Invention

This invention relates to applicators for applying adhesives and sealants, including biomedical adhesives and sealants, methods of making them, and methods of applying such adhesives and sealants. More particularly, this invention relates to methods of applying a bioactive agent, polymerization rate modifier, and/or polymerization initiator to an applicator tip; applicators and applicator tips produced by such methods; and methods of using the applicators in medical, surgical, and other topical applications. Such applicator tips; applicators and methods find use in, for example, professional, industrial, and home uses.

2. Description of Related Art

The most widely-used products in primary use for home or over-the-counter use for covering or sealing wounds are bandages, which typically include a pad that is taped over the wound. However, bandages pose a range of issues and concerns, such as staying in place after being set and allowing foreign matter to contact the wound, the need to repeatedly change the bandage, and the like.

Products in primary use for wound closure are surgical sutures and staples. Both sutures and staples are recognized to provide adequate wound support. However, both sutures and staples cause additional trauma to the wound site (by reason of the need for the needle and suture or staple to pass through tissue and the need to anesthetize the wound area via needle puncture) and are time-consuming to place, and, at skin level, can cause unattractive wound closure marks. Both sutures and staples are especially problematic in pediatric cases where the patient may have a strong fear response and refuse to cooperate with their placement, and in geriatric cases where the skin tissue is weaker and prone to tearing.

As an alternate to the above treatment methods, adhesives have been proposed for use in wound closure. Similarly, adhesives have been proposed for use in wound covering and protection in such topical applications as surface lacerations, cuts, scrapes, abrasions, stomatitis, and other open surface wounds. One group of such adhesives is the monomeric forms of α-cyanoacrylates.

Typically, for wound closure, the cyanoacrylate adhesive is applied to one or both surfaces of a wound or incision, including the internal portions of the wound, with any excess adhesive being quickly removed from the bonding surfaces. Subsequently, the edges of the wound are held together until they adhere. For example, see U.S. Pat. No. 3,559,652 to Coover, Jr. et al. An additional method of application of the cyanoacrylate surgical adhesive to wounds or incisions involves the formation of a bridge over the wound site. As described in U.S. Pat. No. 3,667,472 to Halpern, incised tissues are held together and maintained in fixed relationship until a cyanoacrylate adhesive has been applied over the incision and allowed the necessary time to develop a bond.

Typically, when used in medical applications, cyanoacrylate adhesives are applied in monomeric form to the surfaces to be joined, sealed, or otherwise treated. Typically, in situ anionic polymerization of the monomer occurs, giving rise to the desired adhesive bond or covering. In these instances, moisture and/or proteins naturally present in the treated tissues initiate polymerization of the adhesives.

An effort has been made to control the rate at which polymerization occurs such that polymerization will occur rapidly enough to be convenient for the user, but not so rapidly that tissue damage occurs due to the polymerization reaction. To control the rate at which the adhesives polymerize (and to improve the shelf life), additives have been included in the monomer adhesive compositions. For example, cyanoacrylate polymerization inhibitors or stabilizers including Lewis acids, such as sulfur dioxide, nitric oxide, boron trifluoride, and other acidic substances, including hydroquinone monomethyl ether, hydroquinone, nitrohydroquinone, catechol, and hydroquinone monoethyl ether have been used. Such inhibitors are disclosed in, for example, U.S. Pat. No. 3,559,652 to Banitt, the subject matter of which is incorporated herein by reference. The addition of these inhibitors and stabilizers inhibits premature polymerization of the monomer and slows down the rate of polymerization once the composition is in contact with the tissue to be treated.

Although it is known to add polymerization inhibitors and stabilizers to cyanoacrylate compositions to increase stability and shelf life of the compositions, the addition of polymerization initiators or accelerators to the cyanoacrylate compositions is not widely performed. Polymerization typically occurs in situ without the need for an external initiator or accelerator. In the situations where an initiator or accelerator is added to the composition, such as when tissue fluids have been removed from the application site, the initiator or accelerator is not added until immediately prior to application of the adhesive. For example, U.S. Pat. No. 4,042,442 to Dombroski et al. discloses the addition of a polymerization initiator (either caffeine or theobromine) to a cyanoacrylate adhesive composition. The caffeine or theobromine is added to the adhesive composition in one of two ways. In the first way, the caffeine or theobromine can be mixed with the cyanoacrylate adhesive composition by stirring just prior to application of the adhesive to the substrates to be joined. In the second way, the caffeine or theobromine is dissolved in a volatile solvent, applied to the surfaces to be joined, the volatile solvent is allowed to evaporate, and then the cyanoacrylate adhesive composition is applied to the surfaces of the substrates to be joined. Both of these methods, while effective, are inconvenient for the user because two separate solutions or two separate applications are required.

In an effort to address this inconvenience and lack of control over the polymerization process, commonly assigned U.S. Pat. No. 5,928,611 (corresponding to earlier-published PCT Application No. WO 96/40797), the disclosure of which is hereby incorporated in its entirety, discloses the incorporation of a polymerization initiator or polymerization rate modifier on an applicator tip. Incorporation of the initiator or the rate modifier into the applicator tip provides convenience because only a single composition is required, and allows a level of control over the polymerization rate that cannot be achieved through reliance on polymerization initiators or rate modifiers naturally present at the wound site (such as water).

The polymerization initiators and/or rate modifiers are incorporated into the applicator tip by spraying, dipping, or brushing the pre-formed applicator tip with a solvent (also referred to herein as a liquid medium) containing the initiator and/or rate modifier. Low boiling point solvents (such as acetone and ethanol, or mixtures thereof) are used to apply the initiator and/or rate modifier.

The applicator tips disclosed in this commonly assigned patent effectively and conveniently permit mixing of a cyanoacrylate composition with a polymerization initiator or a polymerization rate modifier during dispensing. The polymerization reaction that ensues, however, can be highly exothermic, and, like other methods currently in use, can cause tissue damage at the site of application due to excessive heat generation during polymerization.

In addition to adding polymerization inhibitors, stabilizers, and initiators to monomeric cyanoacrylate compositions, it is also known to add bioactive materials to these adhesive compositions. Often, these bioactive materials are medicaments which are added to the adhesive compositions to aid in the healing process when the cyanoacrylate adhesives are used to close wounds. For example, U.S. Pat. No. 5,684,042 to Greffet al. discloses a cyanoacrylate composition comprising an antimicrobially-effective amount of an iodine-containing antimicrobial agent; U.S. Pat. Nos. 5,514,371 and 5,624,669 to Leung, et al. disclose the addition of a therapeutic agent in a cyanoacrylate composition; U.S. Pat. No. 4,940,579 to Randen discloses a composition comprising a medicament and a cyanoacrylate adhesive; U.S. Pat. No. 5,254,132 to Barley et al. discloses the use of cyanoacrylate adhesives in conjunction with antibiotics; U.S. Pat. No. 5,866,106 to Papay discloses the addition of vitamins and minerals in a cyanoacrylate composition; and commonly assigned U.S. Pat. No. 6,352,704 discloses monomeric adhesive composition comprising a polymerizable 1,1-disubstituted ethylene monomer and a flavoring additive.

While all of these methods include combining cyanoacrylate adhesives with bioactive materials, the disclosed methods are inconvenient for applying adhesive compositions because multiple solutions and/or applicators are required in order to mix the initiator and adhesive composition or fail to provide a way of controlling the rate at which polymerization proceeds. Furthermore, the selection of bioactive materials has generally been limited by the desire to avoid interaction between the adhesives and the bioactive materials.

Parent application Ser. No. 09/430,177 discloses methods for applying a bioactive agent, polymerization rate modifier, and/or polymerization initiator to an applicator tip. The method uses methanol, alone or as a component of a mixture of low boiling point solvents, as a carrier medium to apply the material. The method provides an unexpectedly superior distribution profile of the material on, and within, the applicator tip, which in turn allows a reduction in polymerization time of the dispensed monomeric adhesive while avoiding tissue damage due to the highly exothermic polymerization reaction.

Despite these known methods for applying an initiator or other material to an applicator tip, the need continues to exist for improved methods for applying the material, and for improved applicators produced thereby.

### SUMMARY OF THE INVENTION

According to the present invention, an applicator is provided where a desired material, such as a polymerization and/or cross-linking initiator or rate modifier, is applied to or incorporated into an applicator tip when the bulk material of the applicator tip is initially formed. It has been discovered that such an applicator tip provides superior performance characteristics, as well as other benefits. For example, as compared to an applicator tip produced by prior methods where the material is applied to a pre-formed tip, the applicator tips of the present invention have been found to provide the same effects (such as polymerization initiation) with a significantly lesser amount of the material. In addition, the applicator tips of the present invention have a more desirable appearance and texture, being softer to the touch, not as coarse, and having a better appearance.

Applying or incorporating the material when the applicator tip is initially formed has been found to provide an unexpectedly superior distribution profile of the material on, and within, the applicator tip. The superior distribution profile allows a reduction in polymerization time of the dispensed monomeric adhesive while avoiding tissue damage due to the highly exothermic polymerization reaction. It has also been discovered that bioactive materials and/or flavorants, which can be polymerization initiators and/or rate modifiers as well, can be applied to applicator tips, providing improved convenience when treating a tissue. Such applicator tips can be used, for example, to apply monomer-containing adhesive compositions to a desired surface.

According to the present invention, the term “applicator tip” is used herein to refer to a wide range of structures, which form part or all of an applicator product. The applicator tips herein refer to structures that are used to directly or indirectly apply a polymerizable material to a substrate, and wherein a selected material is incorporated into the structure of the applicator tip. Thus, for example, the applicator tips described herein can be similar to those described in U.S. Pat. Nos. 5,928,611, 6,099,807, 6,322,852, and 6,376,019, the entire disclosures of which are incorporated herein by reference, such as where the applicator tip is placed in the end of an applicator device that contains the polymerizable material, and where the polymerizable material is expressed through the applicator tip. Likewise, the applicator tips described herein can be similar to those described in U.S. Pat. Nos. 6,283,933 and 6,595,940, the entire disclosures of which are incorporated herein by reference, such as where the applicator tip is a material placed on the end of a swab (which can be solid or hollow). Still further, the applicator tips described herein can be similar to those described in pending U.S. patent application Ser. No. 10/649,879, filed Aug. 28, 2003, the entire disclosures of which are incorporated herein by reference, such as where the applicator tip is a pad placed on one side of a strip member and which pad is used to apply a polymerizable monomner. Other variations of the applicator tip and applicator are described in the above-cited patents and applications, as well as in the following discussion, and are likewise encompassed by the term “applicator tip” herein.

In one aspect, the present invention provides a method of applying at least one material to an applicator tip used to dispense and/or apply liquid compositions. In embodiments, the material may be applied to or incorporated into the applicator during formation of the applicator tip itself. Thus, for example, the material can be incorporated directly into the applicator tip during blow molding, reaction molding, foam formation, or the like, of the material forming all or part of the applicator tip structure. The subject part of the applicator tip structure can be, for example, an applicator tip, a swab tip, a pad, or the like, which is used to subsequently dispense or apply a polymerizable adhesive material.

Thus, in aspects of the present invention, the material is incorporated into the applicator tip during the manufacturing process of the applicator tip. In embodiments, a desired distribution profile of the material on, and/or within, the applicator tip can be achieved without the need for an extra step of applying the material to an already formed applicator tip.

In embodiments, the material is applied to an applicator tip such that the material is present in the applicator tip in a homogeneous, uniform or substantially uniform distribution profile.

In embodiments, the material is an initiator and/or a rate modifier for polymerization and/or cross-linking of a polymerizable monomer. As used herein, a polymerization initiator is any material that causes a monomer composition applied to a substantially dry tissue (i.e., substantially in the absence of plasma or like tissue fluids) to polymerize in less than 300 seconds at ambient temperature, for example, at approximately 21-25° C. Preferably, the initiator causes the monomer composition to polymerize in less than 150 seconds at ambient temperature, more preferably within 130 seconds. As used herein, a polymerization rate modifier is any material that changes the rate at which a polymerizable monomer would polymerize in the absence of that material. Preferably, the rate modifier accelerates the rate of the polymerization reaction.

In embodiments, the initiator or rate modifier is an accelerator or catalyst. In embodiments, the initiator and/or rate modifier is bioactive. In other embodiments, the material applied to the tip is bioactive or a flavorant, but not an initiator or rate modifier for polymerization and/or cross-linking of the polymerizable monomer.

The present invention also provides a method of using an applicator tip containing a polymerization and/or cross-linking initiator, a polymerization and/or cross-linking rate modifier, and/or a bioactive material and/or a flavorant to apply a monomeric composition to a desired site, such as a wound, a surgical site, or any other topical or deep tissue site. In embodiments, the method is used to treat wounds or to treat or protect topical sites, such as areas of skin prone to wounding.

The present invention also provides a method of delivering a bioactive material to a tissue. As used herein, tissue includes any tissue of a human or animal, such as skin, mucous membranes, oral/nasal tissues, gastrointestinal tissues, organ tissues, tumors, non-keratinous tissues, etc.

Applicator tips according to the present invention provide several advantages, including the ability to:

a) control the molecular weight distribution of the polymerized or cross-linked adhesive;

b) control the setting time of the polymerized or cross-linked adhesive;

c) provide precision and convenience in applying the adhesive to a tissue;

d) extend the shelf life of the monomer;

e) reduce the amount of unreacted monomer at the completion of the polymerization reaction, thus avoiding associated monomer odors after polymerization;

f) control the flow properties of applied cyanoacrylate adhesives;

g) provide a bioactive material and/or flavorant to a wound site while simultaneously providing wound closure, protection, and/or coverage; and/or

h) combinations thereof.

### DETAILED DESCRIPTION OF PREFERRED EMBODIMENTS

According to the present invention, the initiator and/or other material is incorporated into the applicator tip during the manufacturing process of the structural material of the applicator tip. Preferably, the initiator and/or other material is incorporated into the applicator tip by mixing or otherwise dispersing the initiator and/or other material with the precursor reactants used to make the applicator tip. That is, the initiator and/or other material is part of the chemical mixture that is used to form the applicator tip, such as by reaction, foaming, molding or the like.

In one embodiment of the present invention, the applicator tip is formed of a foam material, such as a polyurethane foam. As is conventionally known in the art, such foams can be made by a variety of methods, including blow-molding (where a blowing agent is used to expand the reacting materials in a mold), and non-blow molding (where a blowing agent is not used). In such conventional methods, the foam is typically formed by reacting the precursor materials, optionally in the presence of a catalyst, water, and/or other additives, to form the foam material. For example, a polyurethane foam can be formed by reacting a polyol component with an isocyanate component, optionally in the presence of a catalyst, water, and/or other additives. According to the present invention, the conventional foam production method is modified to include the material of the present invention (i.e., bioactive agent, polymerization rate modifier, and/or polymerization initiator) along with the reactants, such that the material is incorporated into the bulk material of the foam during foam production. Such material can be included in addition to the conventional components, or in place of one or more conventional components. For example, the material can replace some or all of the catalyst and/or water that is conventionally used in foam production.

In other embodiments of the present invention, the material can be incorporated into pellets or granules that are molded to form the applicator tip; and the like.

For example, where the applicator tip is made by molding pellets of a polymeric substance, the material can be incorporated into the applicator tip prior to or concurrent with molding of the applicator tip. For example, the material can be mixed with the pellets used to form the applicator tip, such that the mixture is molded to form the applicator tip. Alternatively, where the material is a liquid or can be dissolved into a suitable carrier liquid, the material can be absorbed into or adsorbed onto the pellets prior to molding. An advantage of using foams for the applicator tip is that the materials described herein can be incorporated into the foam during the foam formation. The materials can be incorporated into the foam, for example, by introducing them into the foam during the blowing or foam-forming process, and the like. These processes provide alternative means to incorporate the initiator or other material into or onto the applicator tip in a controlled manner, without need for a subsequent step of applying the material to the pre-formed applicator tip.

Any other variations of the production processes can be used, so long as the objectives of the present invention are achieved whereby the material is incorporated into the applicator tip when the applicator tip is produced. These production methods thus avoid the otherwise subsequent steps of impregnating or coating the material into or onto the already formed applicator tip.

Once the desired foam applicator tip material is formed, which includes the desired initiator, bioactive or the like, the foam is preferably in embodiments subjected to a post-formation treatment. For example, in the case of foam applicator tips, the applicator tip can be quenched or zapped to alter the pore structure. In the case of quenching, the foam is immersed in a tank or container filled with a caustic solution that dissolves the cell membranes (i.e., windows) of the foam. Herein the term “caustic” generally refers to caustic soda solutions, such as NaOH, but any similar or equivalent material can also be used. Following immersion of the foam, the foam is washed using a clear water. Once quenched, the foam generally appears duller and more skeletal in appearance. In addition, the quenched foam is softer and is more suitable for filtration purposes, because it is more tortuous. Further details of producing a quenched foam material, which are equally applicable to the present invention, are described in U.S. patent application Ser. No. 09/897,080 filed Jul. 3, 2001, the entire disclosure of which is incorporated herein by reference. In the case of zapped foam, the foam is subjected to reticulation by a thermal method, as is conventionally known in the foam art. In thermal reticulation (or zapping), the foam is placed in a chamber, a vacuum is pulled, and the chamber is filled with a mixture of gases. The gases are ignited and the flame is passed through the foam. This process removes the cell membranes (windows) giving the strands of the foam a polished surface.

In exemplary embodiments, the applicator tip for dispensing or applying polymerizable monomeric compositions can be any suitable applicator tip, such as a porous polyethylene tip or a foam or fibrous swab, which is attached to an applicator body, such as a butyrate applicator tube or an applicator handle such as a plastic, wood, metal or other suitable material handle or holder.

When the applicator is intended to contain an amount of polymerizable monomeric composition, the applicator body or tube may comprises a conduit or reservoir for the polymerizable monomeric composition. In this embodiment, the applicator tip may be operably connected to the conduit or reservoir, such as by being fitted on an open end of the conduit or overlying the reservoir, so that fluid flowing through the conduit or from the reservoir also flows through the applicator tip. In other embodiments, however, the applicator body may be free of an adhesive reservoir and may be intended to function only as a handle by which to grip the applicator, without itself containing the polymerizable monomeric composition. In these embodiments, for example, the applicator body can be a solid or hollow tube, such as a pipe, stick, rod, dowel, or the like, either straight or contoured. Such embodiments can be, for example, intended to apply a polymerizable monomeric composition by dipping the applicator into the polymerizable monomeric composition or dripping the monomeric composition onto the tip, rather than forcing the polymerizable monomeric composition through the applicator tip from the applicator handle.

A benefit of the present invention, in embodiments, is that the material applied to the applicator tip can be applied in a much more uniform manner as compared to the applicator tips previously described. That is, because the material is introduced during production of the applicator tip itself, the material becomes much more uniformly dispersed in the applicator tip material, rather than forming a gradient or only a surface coating on the applicator tip. This more uniform distribution in turn provides improved polymerization characteristics of the polymerizable material.

A further benefit of the present invention, in embodiments, is that the material applied to the applicator tip can be applied in a smaller amount compared to prior applicator tips, but while achieving the same operational results. Thus, for example, in the case of an initiator applied to an applicator tip by prior methods of dipping the pre-formed applicator tip in a solution of the initiator, and the present method of introducing the initiator during manufacture of the applicator tip material, the amount of initiator used in the present invention can be reduced by at least 33% as compared to the prior methods. In embodiments, the amount of initiator used in the present invention can be reduced by at least 50%, at least 66%, or even by at least 75%, as compared to the prior methods. This means that either the amount of material can be decreased, or that the resultant effects provided by that material (such as polymerization initiation or bioactive effect) can be more specifically tailored.

A still further benefit of the present invention, in embodiments, is that the resultant applicator tip has a better appearance and texture, as compared to applicator tips made by the prior methods. The applicator tip of the present invention, in embodiments, is generally not as coarse and feels better to the touch, as compared to prior applicator tips. This is a particular benefit for medical, either professional or over-the-counter uses, where the applicator is to contact skin or tissue surfaces.

The material applied to the applicator tip can be any material, but is preferably an initiator that initiates polymerization and/or cross-linking of the monomer; a polymerization rate modifier, which modifies the rate of polymerization of the monomer; a bioactive material, such as a medicament; and/or a flavorant.

Particular initiators and rate modifiers for particular monomers may be readily selected by one of skill in the art without undue experimentation. Control of the molecular weight distribution of the applied adhesive can be enhanced by selection of the concentration and functionality of the initiator or rate modifier vis-a-vis the selected monomer. Suitable polymerization initiators and rate modifiers for cyanoacrylate compositions include, but are not limited to, detergent compositions; surfactants, including nonionic surfactants such as polysorbate 20 (e.g., Tween 20™; ICI Americas), polysorbate 80 (e.g., Tween 80™; ICI Americas), and poloxamers; cationic surfactants such as tetrabutylammonium bromide; anionic surfactants, including quaternary ammonium halides such as benzalkonium chloride or its pure components, and benzethonium chloride; stannous octoate (tin (II) 2-ethylhexanoate), and sodium tetradecyl sulfate; and amphoteric or zwitterionic surfactants such as dodecyldimethyl(3-sulfopropyl) ammonium hydroxide, inner salt; amines, imines, and amides, such as imidazole, tryptamine, urea, arginine and povidine; phosphines, phosphites and phosphonium salts, such as triphenylphosphine and triethyl phosphite; alcohols such as ethylene glycol; methyl gallate; ascorbic acid; tannins and tannic acid; inorganic bases and salts, such as sodium bisulfite, magnesium hydroxide, calcium sulfate and sodium silicate; sulfur compounds such as thiourea and polysulfides; polymeric cyclic ethers such as monensin, nonactin, crown ethers, calixarenes and polymeric epoxides; cyclic and acyclic carbonates, such as diethyl carbonate; phase transfer catalysts such as Aliquat™ 336 (General Mills, Inc., Minneapolis, Minn.); organometallics; manganese acetylacetonate; radical initiators and radicals, such as di-t-butyl peroxide and azobisisobutyronitrile; and bioactive compounds or agents.

In preferred embodiments, the initiator may be a bioactive material, including quaternary ammonium halides such as alkylbenzyldimethylammonium chloride (benzalkonium chloride; BAC) its pure components, or mixtures thereof, especially those with an alkyl containing 6-18 carbon atoms; benzethonium chloride; and salts of sulfadiazine. Cobalt napthenate can be used as an accelerator for peroxide.

In preferred embodiments, the initiator may be a bioactive material that possesses antiviral, antimicrobial, antifungal and/or wound healing properties. An example of such a material that possesses polymerization initiation and antiviral, antimicrobial, and/or antifungal properties is Gentian Violet, also known as crystal violet or methylrosaniline chloride. Examples of materials that possess polymerization initiation and wound healing properties also include various zinc complexes and zinc salts, antioxidants such as vitamin E and other vitamins and the like, and copper compounds such as copper chloride, copper sulfate and copper peptides, as described in “Copper: An Essential Element for Life,” ProCyte Corporation, available at http://www.humatech.com/technology.html (Oct. 28, 1999), the entire disclosure of which is incorporated herein by reference. Such materials are particularly preferred because they can serve not only as the polymerization initiator or rate modifier for the cyanoacrylate monomer, they can also provide additional benefits to the wound site, such as antiviral effects, antimicrobial effects and/or antifungal effects or help to promote wound healing.

When present, the zinc compound can be present in various forms, such as zinc salts. For example, suitable zinc compounds include, but are not limited to, zinc salts of cyanoacrylic acid, zinc salts of cyanoacetic acid, zinc salts of dicyanoglutaric acid, zinc salts of rosin, zinc oxide, zinc salts of polycyanoacrylic acid, zinc salts of polyacrylic acid, zinc bacitracin, zinc salicylate, zinc stearate, zinc citrate, zinc lactate, mixtures thereof, and the like. Preferably, the zinc compounds are of Zn²⁺. Incorporation of such zinc compounds into the applied cyanoacrylate composition, either prior to or concurrent with application and/or initiation, is particularly effective in promoting wound healing of leg ulcers, thermal burns, and the like.

In embodiments where an antiviral, antimicrobial and/or antifungal material is used, crystal violet is particularly preferred. Crystal violet has many benefits, particularly when used in conjunction with the adhesive monomer compositions of the present invention, including the benefits of providing a visible color at the site of application, avoidance of tattoo scarring when it is used in combination with the adhesive monomer compositions of the present invention, and ability to be incorporated in the applicator tip in various amounts to provide different results.

The polymerizable and/or cross-linkable material may also contain an initiator and/or a rate modifier which is inactive until activated by a catalyst or accelerator (included within the scope of the term “initiator” as used herein) in the applicator tip. Initiators activated by stimulation such as heat and/or light (e.g., ultraviolet or visible light) are also suitable if the tip and/or applicator is appropriately subjected to such stimulation.

The material (i.e., bioactive material, initiator or rate modifier, or the like) may be incorporated into the applicator tip in any desired effective amount. In the case of an initiator or rate modifier, an effective amount is that amount of initiator or rate modifier that effects polymerization to a gel point on dry tissue in less than 300 seconds, preferably within 150 seconds, and more preferably within 130 seconds, at ambient temperature, such as approximately 21-25° C. In the case of other materials, which are not initiators or rate modifiers, an effective amount of the material is that amount that will provide the desired effect either within the applicator tip or at the application site, as appropriate. Thus, for example, an effective amount of a flavorant would be that amount that provides the desired flavor effect when applied to a desired surface.

In embodiments, the material is incorporated into the applicator tip in an amount of from about 0.1 percent by weight or less, to about 10 percent by weight or more, based on the weight of the applicator tip material. For example, acceptable results can be obtained when the material is present in an amount of from about 1 percent by weight to about 8 percent by weight, and preferably from about 2 percent by weight or about 2.5 percent by weight, to about 7 percent by weight or about 7.5 percent by weight. Other suitable loadings of the material are amounts of about 1, 1.5, 2.0, 2.5, 3.0, 4.0, 5.0, 6.0, and 7.0 percents by weight. Of course, other loadings outside these ranges and values can be used, as appropriate.

In embodiments, the material can be incorporated into the applicator tip materials either neat (by itself), or in combination with one or more other substances. For example, two or more different materials can be incorporated into the applicator tip materials, or the material(s) can be incorporated into the applicator tip in combination with one or more solvents, adjuvents, or the like. In a preferred embodiment, the material can be mixed with water as a solvent, and the resultant mixture incorporated into the applicator tip. In this embodiment, the material/water mixture can replace some of the water that might otherwise be used in a conventional foam manufacturing process. Although an organic solvent can be used to assist dispersion of the material in the applicator tip materials, it is preferred in some embodiments that an organic solvent not be used, and that water be used as the solvent, if necessary.

In embodiments, the initiator and/or the rate modifier can be, but does not have to be, bioactive. In embodiments where the initiator and/or the rate modifier is bioactive, the method of the invention can be used to close, cover, or protect tissue and wounds while simultaneously providing a bioactive material to the tissue or wound.

In embodiments where the initiator is also a bioactive material, the bioactive material is applied into the tip in an amount that is effective to initiate polymerization and to be effective for the biological activity intended (e.g., in a sufficient amount to be antiseptic). The bioactive material is selected in conjunction with the polymerizable monomer to be dispensed such that the bioactive material functions as an initiator or rate modifier for the monomer. During dispensing of the monomer composition, the bioactive material is mixed with the monomer composition. In embodiments, the bioactive material can be released to the tissue to be treated at a constant, or near constant, rate over a period of time while the polymerized composition is in contact with the wound site.

As mentioned above, the bioactive material can, but need not, be a polymerization initiator or rate modifier. Where the bioactive material is not an initiator or a rate modifier, an initiator or rate modifier can also be applied to the tip along with the bioactive material. In embodiments where the applicator tip contains a bioactive material, the bioactive material is solubilized, dissolved, or otherwise dispersed in the adhesive composition as the composition enters and leaves the tip. Thus, the bioactive material similarly mixes with the adhesive composition prior to, and during, application of the adhesive. Coapplication of the bioactive material and adhesive composition allows this mixing to occur. Further mixing can occur once the adhesive composition/bioactive material has been dispensed or applied at the wound site. Such coapplication (e.g., coelution) of the bioactive material and the adhesive composition provides an advantage not disclosed in the prior art.

Suitable bioactive materials include, but are not limited to, medicaments such as antibiotics, antimicrobials, antiseptics, bacteriocins, bacteriostats, disinfectants, steroids, anesthetics, antifungal agents, anti-inflammatory agents, antibacterial agents, antiviral agents, antitumor agents, growth promoting substances, antioxidants, or mixtures thereof. Such compounds include, but are not limited to, acetic acid, aluminum acetate, bacitracin, bacitracin zinc, benzalkonium chloride, benzethonium chloride, betadine, calcium chloroplatinate, certrimide, cloramine T, chlorhexidine phosphanilate, chlorhexidine, chlorhexidine sulfate, chloropenidine, chloroplatinatic acid, ciprofloxacin, clindamycin, clioquinol, cysostaphin, gentamicin sulfate, hydrogen peroxide, iodinated polyvinylidone, iodine, iodophor, minocycline, mupirocin, neomycin, neomycin sulfate, nitrofurazone, non-onynol 9, potassium permanganate, penicillin, polymycin, polymycin B, polymyxin, polymyxin B sulfate, polyvinylpyrrolidone iodine, povidone iodine, 8-hydroxyquinoline, quinolone thioureas, rifampin, rifamycin, copper chloride, copper sulfate, copper peptides, silver acetate, silver benzoate, silver carbonate, silver chloride, silver citrate, silver iodide, silver nitrate, silver oxide, silver sulfate, sodium chloroplatinate, sodium hypochlorite, sphingolipids, tetracycline, zinc oxide, salts of sulfadiazine (such as silver, sodium, and zinc), antioxidants such as vitamins such as vitamin E, other agents mentioned above, and mixtures thereof. Preferable bioactive materials are USP approved, more preferably USP monographed.

As an alternative to using an additional polymerization initiator or rate modifier, it is possible to formulate the applicator such that the adhesive can be initiated by the tip structural material when it is applied to the desired surface. For examples, the applicator tip could be treated with a basic agent, after or preferably before or during its attachment to an applicator body. Treatment with such an agent, such as a caustic agent or alkyl hydroxide, can cause reticulation of the applicator tip material, which in turn results in an applicator tip that is self-initiating when the polymerizable material comes into contact with the applicator tip. In this embodiment, additional polymerization initiators or rate modifiers could be omitted, because a desired initiation and polymerization rate could be selected by proper treatment of the applicator tip material.

In this embodiment, the applicator tip material can be treated with any suitable agent, so long as the objectives of the invention are maintained. Suitable agents include, but are not limited to, caustic soda (NaOH), potassium hydroxide, other hydroxides of light metals, ammonium hydroxide, alkyl hydroxides, caustic alcohol (C₂H₅ONa), silver nitrate, other strongly alkaline materials, mixtures thereof, and the like.

In addition to the above materials, or in place thereof, the applicator tip can also include various other materials that may or may not act as a polymerization initiator or rate modifier. For example, the applicator tip can include a flavorant, such that it imparts a flavor to the adhesive material when the adhesive material is applied to a surface. Incorporation of a flavorant is particularly preferred, for example, when the cyanoacrylate adhesive material is to be applied to oral surfaces, such as to treat stomatitis or cold sores. Flavoring additives suitable for use in the present invention are also disclosed in U.S. Pat. No. 6,352,704, the entire disclosure of which is incorporated herein by reference. Although disclosed in the cited patent as being included in the polymerizable adhesive composition itself, the flavoring additives can be incorporated into the applicator tip, as described above.

The present invention is also directed to a method of applying the adhesive composition utilizing an applicator comprising a tip having a polymerization initiator, a polymerization rate modifier, a bioactive material and/or a flavorant therein. According to the invention, any appropriate design for the applicator can be used. Such applicator designs include, but are not limited to, swab applicators, syringes, adhesive guns, pipettes, eyedroppers, vials, and the like with various dispensing nozzles or tips. Suitable applicators may incorporate or be packaged, such as in saleable kits, with one or more containers containing the adhesive composition and/or other components.

For example, the applicator tip may be permanently fixed to or detachable from an applicator container holding the polymerizable and/or cross-linkable material. Such an applicator tip could be attached to the applicator container prior to use and detached from the applicator container subsequent to use in order to prevent premature polymerization or cross-linking of the unapplied material in the applicator container. At this point the applicator tip may be discarded and a new applicator tip may be attached to the applicator container for subsequent use, or the applicator tip may be cleaned and reused.

As a further example, the applicator tip can be a swab attached to a suitable applicator body, such as a plastic, wood, metal or other suitable material handle or holder. Such an applicator can be used, for example, to apply adhesive material from a separate container. The adhesive material can be applied by dipping the swab into the adhesive material, or by otherwise transferring the adhesive material to the swab, and then applying the adhesive material to the desired surface.

In this embodiment, the applicator including the swab tip can be provided separately, or as part of a saleable kit that includes both the applicator and a quantity of adhesive material, which may be either operably connected to the applicator tip or swab, or located in a separate container. Various designs of such kits are disclosed, for example, in U.S. patent application Ser. No. 09/385,030, filed Aug. 30, 1999, the entire disclosure of which is incorporated herein by reference. In such embodiments, the applicator tip can include any or all of the various materials described above. Preferably, the applicator tip in such swab embodiments includes a polymerization initiator or rate modifier that may also be a bioactive material and/or a flavorant.

Additionally, the applicator tip according to the present invention may comprise multiple parts, with at least one part having the initiator, rate modifier, bioactive material and/or flavorant. For example, the component containing the initiator, rate modifier, bioactive material and/or flavorant may be fabricated separately from the other component(s) of the applicator tip and assembled prior to attachment to the applicator body or container.

The applicator tip and the applicator container may be an integral or even monolithic unit. The unit may be preformed as a single piece and charged with polymerizable and/or cross-linkable material. After application of material from the applicator container, the unit may be discarded. Additionally, such an integral or monolithic applicator tip/applicator container unit may be fashioned to provide the capability of recharging the unit with new material as a multiple use device.

The applicator tip may be composed of any of a variety of materials including polymerized materials such as plastics, foams, rubber, thermosets, films, fibers, or membranes. Where foams are used in the applicator tip, the foam can be either an open-celled form, a closed-cell foam, or a mixture thereof. Any suitable foam material can be used and include, for example, thermoplastic polyurethane foam. In swab tip embodiments, the foam is preferably a soft, absorbent thermoplastic polyurethane foam.

In embodiments, the applicator tip may be made from polyurethane, polyesters, polyolefins such as polyethylene, or polyamides. In embodiments, the applicator may be made from polyethylene, such as that sold by Porex Technologies Corp. (Fairburn, Ga.) under the name LabPor®. In embodiments, the applicator tip can also be made from fibers, either natural or synthetic, such as cotton, rayons, nylons, and mixtures thereof. Additionally, the applicator tip may be composed of materials such as metal, glass, paper, ceramics, and the like. The applicator tip material may be porous, absorbent, or adsorbent in nature to enhance and facilitate loading of a material on or in the applicator tip. For example, the applicator tip may be composed of a material having random pores, capillaries, a honeycomb material, a material having a woven pattern, etc. The degree of porosity will depend on the materials being used, and can be determined easily by one of ordinary skill in the art. Porosity is the open volume within the pores of an applicator tip divided by the total volume of the applicator tip.

In embodiments, the applicator tip may be porous and have an average pore size of about 1 μm to about 500 μm. Generally, according to the present invention, an applicator tip having an average pore size of about 1-100 μm such as 10-30 is used with a polymerizable material having a viscosity of about 1-30 cPs, preferably about 2-18 cPs, and more preferably 5-7 cPs at 25° C. An applicator tip having an average pore size of from about 1 μm to about 100 μm is preferably used with a polymerizable material having a viscosity of about 10-30 cPs. When the polymerizable and/or cross-linkable material has a viscosity higher than 7 cPs, the average pore size of the applicator tip is generally increased. For example, an applicator tip having an average pore size of about 100-200 μm such as 140 μm is preferably used with a polymerizable material having a viscosity of about 30-500 cPs, preferably about 35-350 cPs, and more preferably about 200-300 cPs at 25° C. In embodiments, an applicator tip has a porosity of less than or equal to 80 percent.

In embodiments, when using a porous applicator, the amount of initiator or rate modifier necessary to initiate and/or to modify the rate of polymerization and/or cross-linking increases as the pore size of the applicator tip increases.

The applicator tip can have a variety of suitable shapes and sizes. Generally, the dimensional characteristics are limited only by the intended use of the applicator, and practicality considerations. Suitable shapes include, but are not limited to, flat, conical, cylindrical, chisel or polygonal shapes. The length and size of the tip can be varied depending on various application parameters. The tip may be detachable from the applicator body, or may be an integral part of the applicator.

The applicator tip according to the present invention, where it connects to an applicator tube, may have an elongated tubular portion, out of which the mixed polymerizing and/or cross-linking material is expelled. A portion of the applicator tip which is immediately downstream of the applicator tube is advantageously porous in order to avoid a sharp pressure drop and ensure a constant mixed ratio profile. The structure can preferably trap fragments of any barriers or materials used to separate one or more components within the applicator container so that they will not clog the device or contact the patient.

The tip may also include a variety of additives, such as surfactants or emulsifiers. Preferably, the initiator, rate modifier, bioactive material and/or flavorant is soluble or otherwise dispersible in the polymerizable and/or cross-linkable material, and/or comprises or is accompanied by at least one surfactant which helps it co-elute with the polymerizable and/or cross-linkable material. In embodiments, the surfactant may help solubilize it in the polymerizable and/or cross-linkable material. The initiator, rate modifier, bioactive agent and/or flavorant thus mixes with the adhesive composition as the mixture passes through the tip.

The present invention provides a method of wound treatment, including wound closure. The methods of this invention can be used as replacements for, or in addition to, sutures or staples to join together two surfaces by applying the present compositions to opposing wound surfaces that are then held together while polymerization proceeds. The methods of this invention can also be used to coat, protect, or otherwise cover surface, superficial, or otherwise topical wounds including, but not limited to, minor cuts, scrapes, irritations, compromised skin, superficial lacerations, abrasions, burns, sores, and stomatitis. In these and other embodiments, the methods of this invention can be used as replacements for, or in addition to, conventional bandages, The methods of the invention can also be used on tissues that do not show any signs of tissue damage. For example, the methods can be used to deliver medicaments to a patient through healthy tissue. They can also be used, for example, to locally deliver medicaments to tissues such as tumors or organs.

In embodiments, the methods of the present invention comprise (a) applying a composition of the present invention to a desired application site; (b) allowing the composition to polymerize; and (c) optionally, applying the composition at least once more to the coated application site.

The monomer (including prepolymeric) adhesive composition may include one or more polymerizable monomers. Preferred monomers that may be used in this invention are readily polymerizable, e.g. anionically polymerizable or free radical polymerizable, or polymerizable by zwitterions or ion pairs to form polymers. Such monomers include those that form polymers, that may, but do not need to, biodegrade. Such monomers are disclosed in, for example, U.S. Pat. Nos. 5,328,687, 5,928,611 and 6,183,593, U.S. patent application Ser. No. 09/430,177, filed on Oct. 29, 1999, and U.S. Pat. No. 6,183,593, which are hereby incorporated in their entirety by reference herein.

Preferred monomers include 1,1-disubstituted ethylene monomers, such as α-cyanoacrylates including, but not limited to, alkyl α-cyanoacrylates having an alkyl chain length of from about 1 to about 20 carbon atoms or more, preferably from about 3 to about 8 carbon atoms.

The α-cyanoacrylates of the present invention can be prepared according to several methods known in the art. U.S. Pat. Nos. 2,721,858, 3,254,111, 3,995,641, and 4,364,876, each of which is hereby incorporated in its entirety by reference herein, disclose methods for preparing α-cyanoacrylates.

Preferred α-cyanoacrylate monomers used in this invention include methyl cyanoacrylate, ethyl cyanoacrylate, n-butyl cyanoacrylate, 2-octyl cyanoacrylate, methoxyethyl cyanoacrylate, ethoxyethyl cyanoacrylate, dodecyl cyanoacrylate, 2-ethylhexyl cyanoacrylate, butyl cyanoacrylate, 3-methoxybutyl cyanoacrylate, 2-butoxyethyl cyanoacrylate, 2-isopropoxyethyl cyanoacrylate, 1-methoxy-2-propyl cyanoacrylate, hexyl cyanoacrylate, or dodecylcyanoacrylate.

Other suitable cyanoacrylates for use in the present invention also include, but are not limited to, alkyl ester cyanoacrylate monomers such as those having the formula

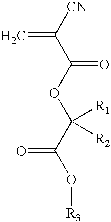

wherein R₁ and R₂ are, independently H, a straight, branched or cyclic alkyl, or are combined together in a cyclic alkyl group, and R₃ is a straight, branched or cyclic alkyl group. Preferably, R₁ is H or a C₁, C₂ or C₃ alkyl group, such as methyl or ethyl; R₂ is H or a C₁, C₂ or C₃ alkyl group, such as methyl or ethyl; and R₃ is a C₁-C₁₆ alkyl group, more preferably a C₁-C₁₀ alkyl group, such as methyl, ethyl, propyl, butyl, pentyl, hexyl, heptyl, octyl, nonyl or decyl, and even more preferably a C₂, C₃ or C₄ alkyl group. Such alkyl ester cyanoacrylates and other suitable monomers are disclosed in, for example, U.S. patent application Ser. No. 09/630,437, filed Aug. 2, 2000, and Ser. No. 09/919,877, filed Aug. 2, 2001, the entire disclosures of which are incorporated herein by reference.

Examples of preferred alkyl ester cyanoacrylates include, but are not limited to, butyl lactoyl cyanoacrylate (BLCA), butyl glycoloyl cyanoacrylate (BGCA), ethyl lactoyl cyanoacrylate (ELCA), and ethyl glycoloyl cyanoacrylate (EGCA). BLCA may be represented by the above formula, wherein R₁ is H, R₂ is methyl and R₃ is butyl. BGCA may be represented by the above formula, wherein R₁ is H, R₂ is H and R₃ is butyl. ELCA may be represented by the above formula, wherein R₁ is H, R₂ is methyl and R₃ is ethyl. EGCA may be represented by the above formula, wherein R₁ is H, R₂ is H and R₃ is ethyl.

The composition may optionally also include at least one other plasticizing agent that assists in imparting flexibility to the polymer formed from the monomer. The plasticizing agent preferably contains little or no moisture and should not significantly affect the stability or polymerization of the monomer. Examples of suitable plasticizers include but are not limited to tributyl citrate, acetyl tri-n-butyl citrate (ATBC), polymethylmethacrylate, silicone oils, siloxanes, and others as listed in U.S. Pat. No. 6,183,593, the disclosure of which is incorporated in its entirety by reference herein. Specific examples of the silicone oils and siloxanes include, for example, but are not limited to, polydimethylsiloxane, hexadimethylsilazane.

The composition may also optionally include at least one thixotropic agent. Suitable thixotropic agents are known to the skilled artisan and include, but are not limited to, silica gels such as those treated with a silyl isocyanate, and optionally surface treated titanium dioxide. Examples of suitable thixotropic agents and thickeners are disclosed in, for example, U.S. Pat. No. 4,720,513, and U.S. Pat. No. 6,310,166, the disclosures of which are hereby incorporated in their entireties by reference herein.

The composition may optionally also include thickeners. Suitable thickeners may include poly (2-ethylhexyl methacrylate), poly(2-ethylhexyl acrylate) and others as listed in U.S. Pat. No. 6,183,593, the disclosure of which is incorporated by reference herein in its entirety.

The composition may also optionally include at least one natural or synthetic rubber to impart impact resistance. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties by reference herein.

The composition may optionally also include one or more stabilizers, preferably both at least one anionic vapor phase stabilizer and at least one anionic liquid phase stabilizer. These stabilizing agents may inhibit premature polymerization. Suitable stabilizers may include those listed in U.S. Pat. No. 6,183,593, the disclosure of which is incorporated by reference herein in its entirety. Furthermore, certain stabilizers may also function as active agents, such as, for example, various acidic anti-microbials, as identified above.

The stability, and thus the shelf-life, of some monomeric adhesive compositions can be further enhanced and extended through careful regulation of the packaging. Treated (e.g., fluorinated polymer) packaging such as that disclosed in copending U.S. patent application Ser. No. 09/430,289, filed Oct. 29, 1999, which is hereby incorporated by reference herein in its entirety, is preferred and may reduce the amount of stabilizer that is combined into the composition. As mentioned above, certain stabilizers including, but not limited to, certain acidics can also function as active agents. In this case, the amount of the active agent/stabilizer material is either not reduced below a level to provide the desired effect, or a further active agent/non-stabilizing agent is added to ensure that the desired effect is provided.

The compositions may also include pH modifiers to control the rate of degradation of the resulting polymer, as disclosed in U.S. Pat. No. 6,143,352, the entire disclosure of which is hereby incorporated by reference herein in its entirety.

Compositions of the present invention may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of formaldehyde scavenger compounds useful in this invention include sulfites; bisulfites; mixtures of sulfites and bisulfites, etc. Additional examples of formaldehyde scavenger compounds useful in this invention and methods for their implementation can be found in U.S. Pat. Nos. 5,328,687, 5,514,371, 5,514,372, 5,575,997, 5,582,834 and 5,624,669, all to Leung et al., which are hereby incorporated herein by reference in their entireties.

To improve the cohesive strength of adhesives formed from the compositions of this invention, difunctional monomeric cross-linking agents may be added to the monomer compositions of this invention. Such crosslinking agents are known. U.S. Pat. No. 3,940,362 to Overhults, which is hereby incorporated herein in its entirety by reference, discloses exemplary cross-linking agents.

The compositions of this invention may further contain fibrous reinforcement and colorants such as dyes, pigments, and pigment dyes. Examples of suitable fibrous reinforcement include PGA microfibrils, collagen microfibrils, and others as described in U.S. Pat. No. 6,183,593, the disclosure of which is incorporated by reference herein in its entirety.

Other compositions that are contemplated by the present invention are exemplified by U.S. Pat. Nos. 5,624,669; 5,582,834; 5,575,997; 5,514,371; 5,514,372; and 5,259,835; the disclosures of all of which are hereby incorporated in their entirety by reference.

In embodiments, any or all of the applicator tip, the applicator with the applicator tip attached, a monomer composition and/or packaging for the various components can be sterilized, if desired. Furthermore, whether or not the applicator tip, applicator and/or composition and container are sterilized, the various materials can further include one or more suitable preservative, if desired.

Sterilization of the applicator tip, applicator, monomer composition and/or its packaging can be accomplished by techniques known to the skilled artisan, and is preferably accomplished by methods including, but not limited to, chemical, physical, and/or irradiation methods. Examples of chemical methods include, but are not limited to, exposure to ethylene oxide or hydrogen peroxide vapor. Examples of physical methods include, but are not limited to, sterilization by heat (dry or moist) or retort canning. Examples of irradiation methods include, but are not limited to, gamma irradiation, electron beam irradiation, and microwave irradiation. A preferred method is electron beam irradiation, as described in U.S. Pat. No. 6,143,805, the entire disclosure of which is incorporated herein by reference. The composition should also show low levels of toxicity to living tissue during its useful life. In preferred embodiments of the present invention, the composition is sterilized to provide a Sterility Assurance Level (SAL) of at least 10⁻³. In embodiments, the Sterility Assurance Level may be at least 10⁻⁴, or may be at least 10⁻⁵, or may be at least 10⁻⁶.

### EXAMPLES

### Comparative Examples 1-9

Polyurethane foam applicator tips are formed by initially forming a polyurethane foam by conventional methods. The foam is zapped (thermally reticulated) and then cut into strips. The strips are applied to solid applicator bodies to form swabs. The applicator tips are next loaded with initiator according to the methods described in U.S. Pat. No. 5,928,611. In particular, a solution is made from varying concentrations of polymerization initiator or a polymerization rate modifier (benzalkonium chloride) in a solvent (acetone). The applicator tips are dipped into the solution. The applicator tip is removed from the solution, and is dried to evaporate the acetone solvent. The process results in varying concentrations of the initiator being deposited in and on the applicator tip, as shown in Table I below in terms of actual initiator weight and percent by weight of the applicator tip.

Four drops of stabilized 2-octyl cyanoacrylate formulation are applied to the initiator-loaded applicators. The polymerization time of the adhesive is measured using a thermocouple probe which is inserted into the foam and is reported in Table 1 below.

TABLE 1

| Initiator Concentration | Initiator Loading on | Polymerization Set Time |
|---|---|---|
| (μg) | Applicator Tip (wt %) | (sec) |
| 0 | 0 | 10000 |
| 120 | 0.8 | 897 |
| 240 | 1.5 | 603 |
| 360 | 2.3 | 435 |
| 480 | 3.0 | 375 |
| 600 | 3.8 | 320 |
| 900 | 5.7 | 265 |
| 1200 | 7.6 | 189 |
| 2160 | 13.7 | 127 |

The data in Table 1 shows that acceptable polymerization set time results are only obtained when the initiator concentration exceeds over about 1000 μg or over about 6 wt % of the applicator tip.

### Examples 1-4

Initiator-loaded applicator tips are produced according to the methods of the present invention. In particular, a polyurethane foam is prepared by conventional methods as in the Comparative Examples, except that varying concentrations of a solution of benzalkonium chloride in water are included in the chemical reactants mix. The reactants are mixed in a reactant bin and poured onto a conveyor. The foam reaction takes place, forming a solid foam on the conveyor. The foam is cut into segments, zapped (thermally reticulated), and then cut into individual applicator tip sized pieces. The process results in varying concentrations of the initiator being uniformly dispersed in the applicator tip, as shown in Table 2 below in terms of actual initiator weight and percent by weight of the applicator tip.

Four drops of stabilized 2-octyl cyanoacrylate formulation are applied to the initiator-loaded applicators tips, and the seeting time is measured as in Comparative Example 1. The polymerization time of the adhesive is reported in Table 2 below.

TABLE 2

| Initiator Concentration | Initiator Loading on | Polymerization Set Time |
|---|---|---|
| (μg) | Applicator Tip (wt %) | (sec) |
| 237 | 1.5 | 91 |
| 316 | 2.0 | 104 |
| 395 | 2.5 | 68.3 |
| 568 | 3.6 | 68 |

The data in Table 2 shows that acceptable polymerization set time results are obtained at all of the tested initiator loading concentrations. Further, a comparison of the results of Tables 1 and 2 demonstrates that improved polymerization set time results are obtained by the present invention, at initiator loading substantially les than in the prior methods.

What is claimed is:

1. A method of making an applicator tip for an adhesive applicator, comprising:
mixing at least one active member selected from the group consisting of bioactive materials, flavorants, polymerization initiators and polymerization rate modifiers with precursor materials of a structural material of an applicator tip, and
reacting the precursor materials to form said structural material of the applicator tip such that said at least one active member is dispersed in said applicator tip.

2. The method of claim 1, wherein said mixing comprises mixing said at least one active member and said precursor materials in a mixing vessel.

3. The method of claim 1, wherein said reacting comprises transferring said mixture to a conveyor belt and allowing a reaction to proceed.

4. The method of claim 1, wherein said precursor materials comprise a polyol and an isocyanate.

5. The method of claim 1, wherein said precursor material is blown in a mold.

6. The method of claim 1, wherein said structural material is a foam.

7. The method of claim 1, wherein said structural material is a porous body.

8. The method of claim 1, wherein said active member is selected from the group consisting of polymerization initiators and polymerization rate modifiers.

9. The method of claim 1, wherein the active member is selected from the group consisting of polysorbate 20, polysorbate 80, poloxamers, tetrabutylammonium bromide, alkylbenzylalkonium chloride, stannous octoate (tin (II) 2-ethylhexanoate), sodium tetradecyl sulfate, and dodecyldimethyl(3-sulfopropyl)ammonium hydroxide.

10. The method of claim 1, wherein the active member is selected from the group consisting of imidazole, tryptamine, urea, arginine, povidine, triphenylphosphine, triethyl phosphite, ethylene glycol, methyl gallate, ascorbic acid, tannins, tannic acid, sodium bisulfite, magnesium hydroxide, calcium sulfate, sodium silicate, thiourea, monensin, nonactin, crown ethers, calixarenes, polymeric epoxides, diethyl carbonate, di-t-butyl peroxide, and azobisisobutyronitrile.

11. The method of claim 1, wherein the active member is alkylbenzyldimethylammonium chloride with an alkyl containing 6-18 carbon atoms, its pure components, or mixtures thereof.

12. The method of claim 1, wherein the active member comprises at least one member selected from the group consisting of antibiotics, antimicrobials, antiseptics, bacteriocins, bacteriostats, disinfectants, steroids, anesthetics, antifungal agents, anti-inflammatory agents, antiviral agents, antitumor agents, and antibacterials.

13. The method of claim 1, wherein the active member comprises a mixture of (i) at least one member selected from the group consisting of polymerization initiators and polymerization rate modifiers, and (ii) at least one member selected from the group consisting of bioactive materials and flavorants.

14. The method of claim 1, wherein the active member comprises at least one compound that is both (i) at least one member selected from the group consisting of polymerization initiators and polymerization rate modifiers and (ii) a bioactive material.

15. The method of claim 14, wherein the active member is selected from the group consisting of antibiotics, antimicrobials, antiseptics, bacteriocins, bacteriostats, disinfectants, steroids, anesthetics, antifungal agents, anti-inflammatory agents, and antibacterials.

16. The method of claim 1, wherein the active member comprises at least one flavorant.

17. The method of claim 16, wherein the flavorant is selected from the group consisting of 5-fold orange oil, anethole, banana distillate, benzaldehyde, clove oil, cold pressed valencia orange oil, cold pressed grapefruit oil, cold pressed lemon oil, cold pressed lime oil, cucumber distillate, honey distillate, menthol, alkyl salicylates, monosodium glutamate, spearmint, wintergreen, cinnamon, citrus, cherry, apple, peppermint, peppermint oil, peppermint spirit, vanillin, thymol, ethyl vanillin, and mixtures thereof.

18. The method of claim 1, wherein said applicator tip comprises a porous polyurethane, polyolefin, polyester, or polyamide.

19. The method of claim 1, wherein said applicator tip comprises porous polyethylene.

20. The method of claim 1, wherein said applicator tip comprises polyurethane foam.

21. The method of claim 1, further comprising quenching said structural material of the applicator tip with a caustic solution.

22. The method of claim 1, further comprising thermally reticulating said structural material of the applicator tip.

23. The method of claim 1, wherein said at least one active member is substantially uniformly dispersed in said structural material of the applicator tip.

24. The method of claim 1, further comprising sterilizing said applicator tip.

25. An applicator for a polymerizable adhesive, comprising an applicator tip made by the method of claim 1, attached to an applicator body.

26. The applicator of claim 25, wherein said applicator body comprises a conduit for a fluid polymerizable adhesive composition, and said applicator tip is operably connected to said conduit so that fluid flowing through said conduit also flows through said applicator tip.

27. The applicator of claim 25, wherein said applicator body comprises a reservoir of a fluid polymerizable adhesive composition, and said applicator tip is operably connected to said reservoir so that fluid from said reservoir will contact said applicator tip.

28. The applicator of claim 25, wherein said applicator body is free of a polymerizable adhesive reservoir.

29. The applicator of claim 28, wherein said applicator tip comprises a foam.

30. The applicator of claim 28, wherein said applicator body is a solid structure.

31. The applicator of claim 26, further comprising a container of polymerizable adhesive physically separated from said applicator tip within said applicator or within a package containing said applicator.

32. The applicator of claim 31, wherein said polymerizable adhesive comprises a 1,1-disubstituted ethylene monomer.

33. The applicator of claim 32, wherein said monomer is an α-cyanoacrylate.

34. The applicator of claim 33, wherein said monomer is selected from the group consisting of butyl and octyl α-cyanoacrylate.

35. An applicator tip made by the process of claim 1.

36. A method of applying a polymerizable or cross-linkable material to a substrate, comprising:
applying said material onto said substrate using an applicator tip made by the process of claim 1.
